(19) United States
(12) Patent Application Publication
(10) Pub. No.: US 2014/0371631 A1
(43) Pub. Date: Dec. 18, 2014

(54) MOBILE DEVICE FOR AN ELECTRONIC STETHOSCOPE INCLUDING AN ELECTRONIC MICROPHONE AND A UNIT FOR DETECTING THE POSITION OF THE MOBILE DEVICE

(71) Applicant: STMicroelectronics S.r.l., Agrate Brianza (IT)

(72) Inventor: Fulvio Vittorio Fontana, Monza (IT)

(21) Appl. No.: 14/305,728

(22) Filed: Jun. 16, 2014

(30) Foreign Application Priority Data
Jun. 17, 2013 (IT) TO2013A000496

(51) Int. Cl. A61B 7/04; H04R 1/46

(52) U.S. Cl. CPC A61B 7/04; H04R 1/46
USPC 600/586

(57) ABSTRACT

A mobile device for an electronic stethoscope, including: a microphone, which receive an acoustic signal coming from an area of the body during a detection period and generates an auscultation signal of an electrical type, as a function of the acoustic signal; a transmitter; a processing unit, which transmits the auscultation signal to an external electronic device, through the transmitter; and an electronic accelerometer, which generates an acceleration signal indicating acceleration of the mobile device. The processing unit generates, on the basis of the acceleration signal, a position signal, indicating the position of the mobile device during the detection period, and transmits the position signal to the external electronic device, through the transmitter.

### BACKGROUND

1. Technical Field

The present invention relates to a mobile device for an electronic stethoscope. In particular, the present invention relates to a mobile device including an electronic microphone and a unit for detecting the position of the mobile device.

2. Description of the Related Art

As is known, in the medical field, stethoscopes of a pneumatic type are commonly used, which facilitate auscultation of body sounds by medical personnel.

Even though they are widely used, pneumatic stethoscopes are characterized by considerable costs and overall dimensions. Moreover, each pneumatic stethoscope is substantially personal, i.e., it is not shared between different subjects, since it comprises portions that, in use, come into direct contact with the auditory canals of the person who uses the stethoscope. In addition, pneumatic stethoscopes do not make it possible to record or share the acoustic signals listened to, or to trace back, once auscultation is over, to the areas of the body that have emitted the acoustic signals previously acquired.

### BRIEF SUMMARY

One or more embodiments are directed to an electronic stethoscope and a mobile device for an electronic stethoscope. In one embodiment, a mobile device includes a first microphone designed to receive an acoustic signal from an area of a body during a detection period and to generate an auscultation signal as a function of the acoustic signal. The mobile device further includes a transmitter and an accelerometer configured to generate an acceleration signal indicating acceleration of the mobile device. The mobile device further includes a processing unit coupled to the first microphone, the transmitter, and the accelerometer. The processing unit is configured to receive the auscultation signal from the first microphone and transmit the auscultation signal to an external electronic device through the transmitter. The processing unit is configured to receive the acceleration signal and generate, based on the acceleration signal, a position signal indicating a position of the mobile device during the detection period, and to transmit said position signal to the external electronic device through the transmitter.

### BRIEF DESCRIPTION OF THE SEVERAL VIEWS OF THE DRAWINGS

For a better understanding of the disclosure, embodiments thereof are now described purely by way of non-limiting example and with reference to the attached drawings, wherein:

FIG. 1 is a schematic illustration of the connections between a mobile device, a cellphone, a portable computer, and the Internet;

FIG. 2 shows a block diagram of an embodiment of the mobile device illustrated in FIG. 1;

FIG. 3 shows a perspective view of the mobile device illustrated in FIG. 1;

FIG. 4 is a cross-sectional view with portions removed of the mobile device illustrated in FIGS. 1 to 3;

FIG. 5 shows a block diagram regarding operations performed by a processing unit of the mobile device illustrated in FIGS. 1 to 4; and

FIG. 6 is a cross-sectional view with portions removed of a further embodiment of the present mobile device.

### DETAILED DESCRIPTION

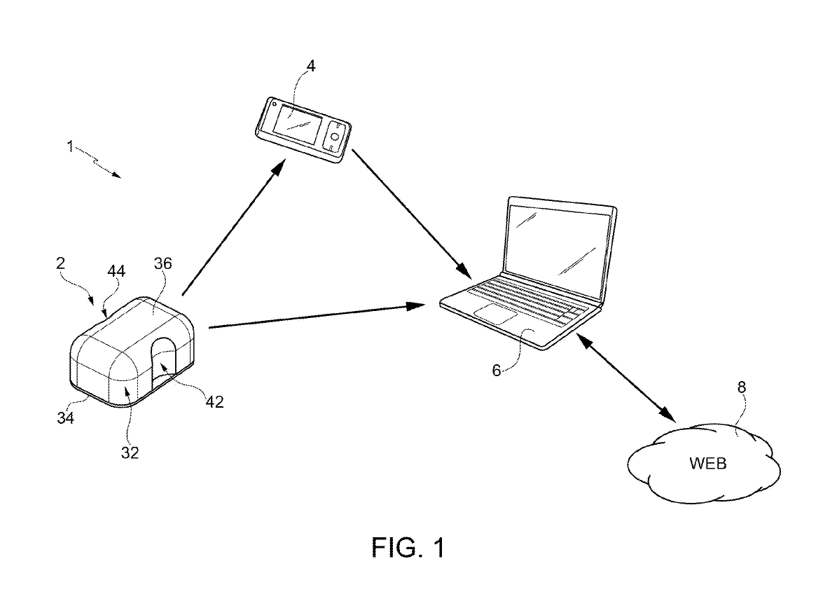

FIG. 1 shows an electronic stethoscope 1, which comprises a mobile device 2, which can be electromagnetically coupled to a cellphone 4 of a multimedia type, also known as smartphone, which combines functions typical of a cellphone with functions typical of a palmtop computer or PDA (Personal Data Assistant). The mobile device 2 can moreover be electromagnetically coupled to a computer 6 of a portable type, connected, in turn, to the Internet 8. Moreover, in a way in itself known, the cellphone 4 can transmit information to the computer 6.

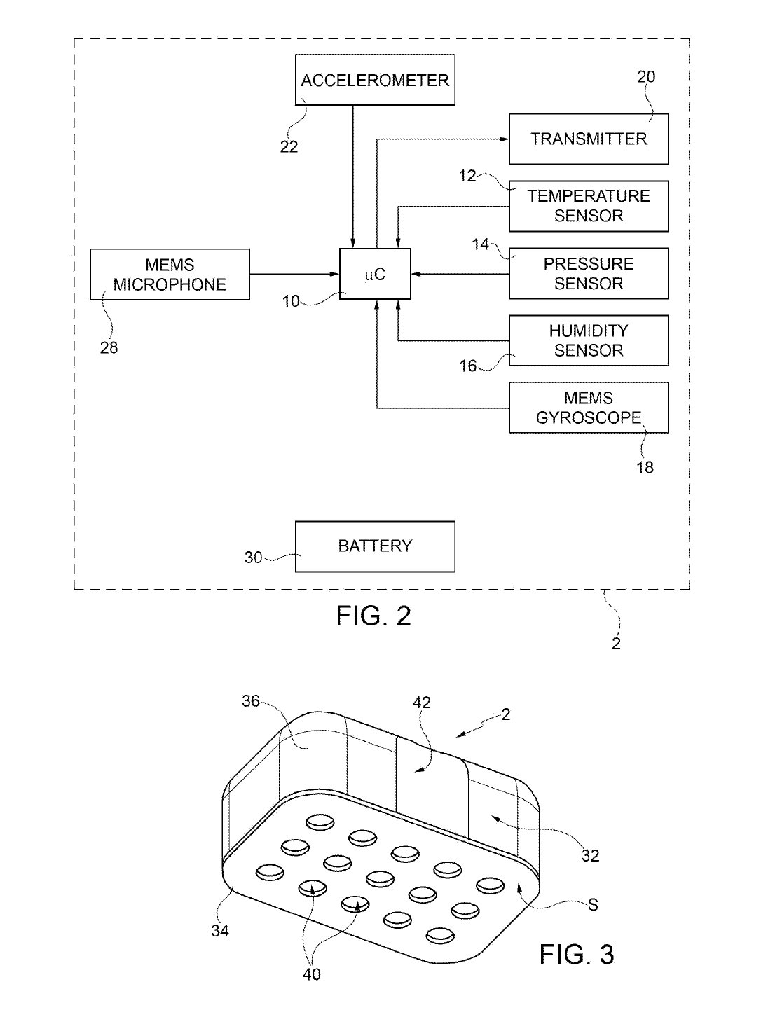

As illustrated in FIG. 2, the mobile device 2 comprises: a processing unit 10, a temperature sensor 12, a pressure sensor 14, a humidity sensor 16, a gyroscope 18, a transmitter 20, and an accelerometer 22. Moreover, the mobile device comprises at least one microphone 28 and a battery 30. In the embodiment illustrated in FIG. 2, the mobile device 2 comprises a single microphone 28.

In greater detail, the temperature sensor 12 is of a type in itself known and is designed to supply to the processing unit 10, to which it is connected, a temperature signal of an electrical type, which indicates the temperature detected by the temperature sensor 12 itself.

The pressure sensor 14 is of a type in itself known and is designed to supply to the processing unit 10, to which it is connected, a pressure signal of an electrical type, which indicates the pressure detected by the pressure sensor 14 itself.

The humidity sensor 16 is of a type in itself known and is designed to supply to the processing unit 10, to which it is connected, a humidity signal of an electrical type, which indicates the humidity detected by the humidity sensor 16 itself.

The gyroscope 18, which is in itself known, is of the MEMS (Micro-Electro-Mechanical Systems) type. Moreover, the gyroscope 18 is triaxial. Hence, it is designed to generate a first angular-position signal, a second angular-position signal, and a third angular-position signal, which are of an electrical type and indicate three angles θᵤ, θᵥ, θₖ, respectively, these three angles being the angles of which the gyroscope 18 and hence the mobile device 2 are rotated with respect to a reference orientation and to the axes of an orthogonal reference system uvk.

The first, second, and third angular-position signals as a whole form an attitude signal, indicating the angular position (attitude) of the mobile device 2 with respect to the reference orientation. Moreover, the gyroscope 18 supplies the first, second, and third angular-position signals to the processing unit 10, to which it is connected.

The transmitter 20, which is in itself known, is of a wireless type, includes an antenna (not illustrated) and is designed to couple the mobile device 2 to the cellphone 4 and to the computer 6, for example, through a data-communication protocol of a known type. In this way, the processing unit 10 can transmit signals to the cellphone 4 and to the computer 6, through the transmitter 20, as described hereinafter.

The accelerometer 22, which is in itself known, is of a MEMS type and is triaxial. Hence, it is designed to generate a first acceleration signal, a second acceleration signal, and a third acceleration signal, which are of an electrical type and indicate three components aₓ, a_{y}, a_{z} for linear acceleration to which the accelerometer 22 itself and hence also the mobile device 2 are subjected. The three components aₓ, a_{y}, a_{z} refer to three orthogonal axes of a reference system xyz, which may be respectively parallel, for example, to the axes of the aforementioned reference system uvk.

As a whole, the first, second, and third acceleration signals form a vector acceleration signal. Moreover, the accelerometer 22 supplies the first, second, and third acceleration signals to the processing unit 10, to which it is connected.

The microphone 28, which is in itself known, is of a MEMS type and supplies to the processing unit 10, to which it is connected, an auscultation signal of an electrical type, which indicates an acoustic signal, i.e., the pressure wave detected by the microphone 28 itself during a corresponding detection period.

The battery 30 is connected to the processing unit 10, to the temperature sensor 12, to the pressure sensor 14, to the humidity sensor 16, to the gyroscope 18, to the transmitter 20, to the accelerometer 22, and to the microphone 28, even though these connections are not illustrated in FIG. 2, for simplicity of representation. The battery 30 is of a rechargeable type, by electrical coupling to a power supply (not illustrated).

As illustrated in FIG. 1 and in FIG. 3, the mobile device 2 comprises a shell 32 formed by a first portion 34 and a second portion 36.

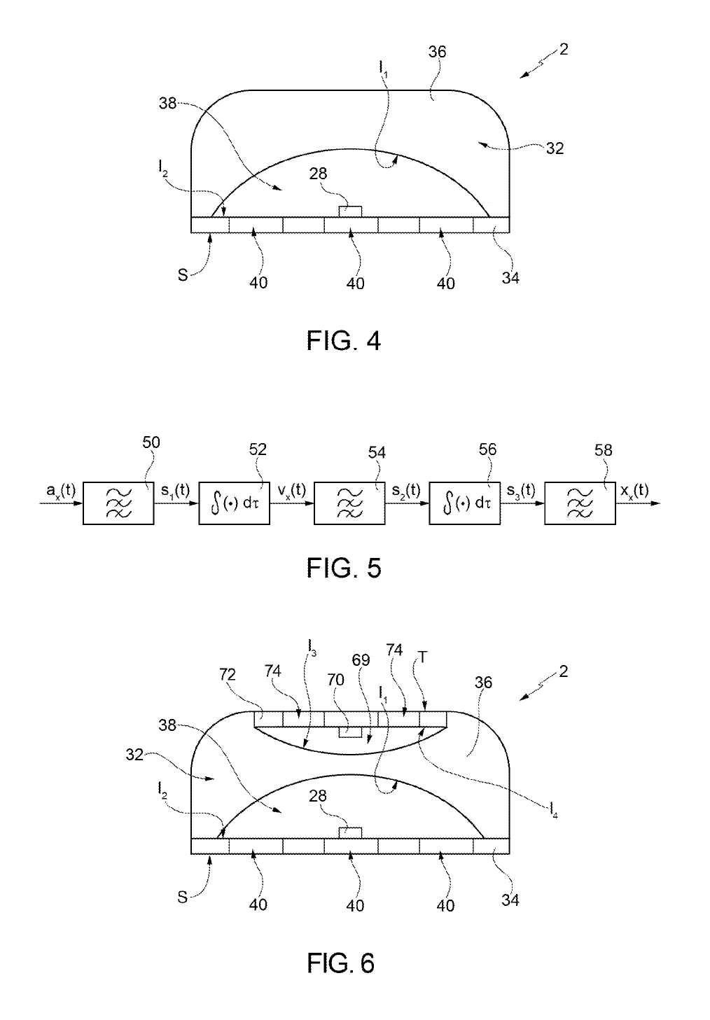

The first and second portions 34, 36 are made, for example, of plastic material and are mechanically coupled so as to form a cavity 38 (FIG. 4), arranged inside which is a printed circuit board (PCB) (not illustrated), which carries the processing unit 10, the temperature sensor 12, the pressure sensor 14, the humidity sensor 16, the gyroscope 18, the transmitter 20, the accelerometer 22, the microphone 28, and the battery 30.

As illustrated in FIG. 4, the second portion 36 forms a first internal surface I₁, whereas the first portion 34 forms a second internal surface I₂, which faces the first internal surface I₁. The first and second internal surfaces I₁, I₂ delimit the cavity 38.

In greater detail, the first portion 34 has, for example, a plane shape and has a plurality of holes 40 of a through type, which enable the acoustic signals to penetrate into the cavity 38 so as to be detected by the microphone 28. In practice, the holes 40 set the cavity 38 in fluid communication with the outside world.

The first portion 34 moreover defines, in addition to the aforementioned second internal surface I₂, a contact surface S, which is parallel to the second internal surface I₂ and is designed to contact the areas of the body on which auscultation is to be carried out; the holes 40 extend between the second internal surface I₂ and the contact surface S.

The contact surface S may be coated with a disposable adhesive film (not illustrated), which has a low acoustic impedance and can be removed after use, for hygienic reasons.

On the outside, the second portion 36 is shaped like a cap and can be conveniently gripped by the person that carries out auscultation. In particular, in the embodiment illustrated in FIGS. 1 and 3, the second portion 36 of the shell 32 forms a first recess 42 and a second recess 44, inside which the person who carries out auscultation can set the tips of his or her thumb and index finger in order to move the shell 32.

As illustrated once again in FIG. 4, where, for simplicity of representation, illustrated inside the cavity 38 is just the microphone 28, the first internal surface I₁ is shaped like a paraboloid. Moreover, the microphone 28 is arranged substantially in the focus of the paraboloid so as to optimize coupling between the acoustic signals, which traverse the holes 40 and are reflected by the first internal surface I₁ and the microphone 28 itself. The latter aspect is not, however, illustrated in FIG. 4, where the position of the focus with respect to the first internal surface I₁ is purely qualitative, as on the other hand also the plotting of the first internal surface I₁ itself.

In use, the processing unit 10 transmits the auscultation signal generated by the microphone 28 to the cellphone 4 and to the portable computer 6, through the transmitter 20. Moreover, the processing unit 10 transmits to the cellphone 4 and to the portable computer 6 the temperature signal, the pressure signal, and the humidity signal generated, respectively, by the temperature sensor 12, the pressure sensor 14, and the humidity sensor 16.

Referring for convenience to the person carrying out auscultation as “the user”, the user can then store the auscultation signal, the temperature signal, the pressure signal, and the humidity signal in the cellphone 4 and/or in the computer 6. Moreover, the user or another person can listen to the auscultation signal through the speaker of the cellphone 4 and/or the speaker of the computer 6. Listening to the auscultation signal can hence be carried out in a period of time different from the period in which the auscultation signal has been acquired. In addition, the auscultation signal can be listened to an unlimited number of times. The user can moreover display the aforementioned temperature, pressure, and humidity signals on the cellphone 4 and/or on the computer 6, and can then display the values of the corresponding quantities.

The processing unit 10 is moreover configured so as to determine a first linear-velocity signal, a second linear-velocity signal, and a third linear-velocity signal, which indicate corresponding components of the instantaneous linear velocity of the mobile device 2. Moreover, the processing unit 10 is configured so as to determine a first linear-position signal, a second linear-position signal, and a third linear-position signal, which indicate the components of a vector that identifies the linear position of the mobile device 2 with respect to a reference point.

In detail, the first linear-velocity signal and the first linear-position signal are determined based on the first acceleration signal, as described in greater detail in what follows. Likewise, the second linear-velocity signal and the second linear-position signal are determined based on the second acceleration signal, whereas the third linear-velocity signal and the third linear-position signal are determined based on the third acceleration signal.

In greater detail, assuming, for example, that we are referring to the first acceleration signal, in order to determine the first linear-velocity signal and the first linear-position signal, the processing unit 10 carries out the operations illustrated in FIG. 5, where the first acceleration signal, the first linear-velocity signal, and the first linear-position signal are designated, respectively, by aₓ(t), vₓ(t) and xₓ(t).

The processing unit 10 carries out a filtering (block 50) of a high-pass type on the first acceleration signal aₓ(t) so as to remove possible effects of drift of the accelerometer 22, to obtain a first intermediate signal s₁(t).

The processing unit 10 carries out integration in time (block 52) of the first intermediate signal s₁(t), to obtain the first linear-velocity signal vₓ(t).

The processing unit 10 carries out a filtering (block 54) of a high-pass type on the first linear-velocity signal vₓ(t) so as to remove the d.c. component of the first linear-velocity signal vₓ(t), to obtain a second intermediate signal s₂(t).

The processing unit 10 carries out a filtering (block 58) of a high-pass type on the third intermediate signal s₃(t), to obtain the first linear-position signal xₓ(t). In practice, assuming having positioned, at a first instant t₀, the mobile device 2 on a reference point of the body (for example, the navel), and assuming having subsequently moved the mobile device 2 until it is brought, at a subsequent instant t, into a point of the body to be analyzed, the position of the point of the body to be analyzed is defined, with respect to the position of the reference point of the body, by the aforementioned vector, i.e., by a triad of displacements [Δx Δy Δz], the displacements being expressed, for example, with respect to the reference system xyz. Given this, the first linear-position signal xₓ(t) indicates precisely the displacement Δx; i.e., it provides a measurement of the latter.

Operations that are the same as the operations illustrated in FIG. 5 are performed by the processing unit 10 based on the second and third acceleration signals to determine, respectively, the second linear-velocity signal and the second linear-position signal, and the third linear-velocity signal and the third linear-position signal. The second and third linear-position signals hence indicate, respectively, the displacement Δy and the displacement Δz.

In use, the processing unit 10 hence has available, at each instant, a measurement of the corresponding linear position of the mobile device 2, as well as a measurement of the corresponding attitude. Consequently, the processing unit 10 functions, together with the accelerometer 22 and the gyroscope 18, as unit for detecting the position and attitude of the mobile device 2.

The processing unit 10 transmits the first, second, and third linear-velocity signals, the first, second, and third linear-position signals, and the first, second, and third angular-position signals to the computer 6 and, optionally, to the cellphone 4.

The computer 6 determines a sort of audio-visual map; i.e., it associates, for each detection period, the corresponding auscultation signal to the linear position and to the angular position of the mobile device 2 during the detection period, as measured by the processing unit 10.

In particular, the computer 6 reproduces the acoustic signal detected during each detection period and simultaneously displays the corresponding linear and angular positions of the mobile device 2. In this way, the user can easily identify, given an acoustic signal, the corresponding area of the body from which the acoustic signal has come, as well as the angular position of the mobile device during detection. Simultaneously, the user can display, as mentioned previously, the humidity and temperature of this area of the body, as well as the pressure detected by the pressure sensor 14 during the detection period. The information regarding the pressure values may be used, for example, to determine the position (for example, supine or upright) and/or variations of position of the patient during auscultation. The information regarding the pressure values can then be used for measuring the position of the mobile device 2 along an axis parallel to the direction of the force of gravity.

The computer 6 can moreover use the first, second, and third linear-velocity signals for filtering the auscultation signal so as to remove any possible contributions of acoustic noise caused by rubbing of the mobile device 2 against the skin.

The advantages that the present mobile device affords emerge clearly from the foregoing discussion. In particular, the present mobile device makes it possible to listen to an acoustic signal of the body in a shared way and irrespective of the effective period of acquisition of the corresponding auscultation signal. Moreover, the present mobile device enables generation, through a computer, of an audio-visual map, where each auscultation signal is associated to the corresponding area of the body examined. Advantageously, also the information regarding the temperature and humidity of the area of the body examined is displayed.

Finally, it is evident that modifications and variations may be made to the mobile device described herein, without thereby departing from the scope of the present disclosure.

For example, the gyroscope 18, the accelerometer 22, and the microphone 28 may be of a type different from what has been described. The computer 6 may be of a fixed type.

As mentioned previously, the mobile device 2 may moreover include a number of microphones greater than one, possibly according to the area of the body on which auscultation is to be carried out. A larger number of microphones enables reduction of the time utilized for auscultation of a given area of the body.

In the case where the mobile device 2 includes a plurality of microphones, the first internal surface I₁ may have a shape different from what has been described. For example, the first internal surface I₁ may be designed so as to maximize reflection of the acoustic signals in the direction of the microphones. Consequently, the first internal surface I₁ may be such as to define a plurality of foci, each microphone being arranged in a corresponding focus. For example, the first internal surface I₁ may be formed by a plurality of portions, each portion belonging to a corresponding geometrical surface having a corresponding focus.

One or more from among the temperature sensor 12, the pressure sensor 14, the humidity sensor 16, and the gyroscope 18 may be absent. Moreover, the temperature sensor 12, the pressure sensor 14, and the humidity sensor 16 may be integrated within a multifunction sensor.

With regard to the processing unit 10, before transmitting, through the transmitter 20, one or more from among the auscultation signal, the temperature signal, the pressure signal, the humidity signal, the linear-velocity signals, and the linear-position and angular-position signals, it is possible to carry out a process of amplification and/or filtering.

As illustrated in FIG. 6, the mobile device 2 may moreover envisage, in addition to the microphone 28, an auxiliary device 70, formed, for example, by a microphone that is identical to the microphone 28.

In this case, the shell 32 comprises a third portion 72; furthermore, the second portion 36 forms a third internal surface I₃, having the shape of a paraboloid with concavity opposite to the concavity of the first internal surface I₁, the paraboloids of the first and third internal surfaces I₁, I₃, having, for example, axes that coincide. The third portion 72 is mechanically coupled to the second portion 36, is planar and is delimited by a fourth internal surface I₄, arranged facing the third internal surface I₃, and by a distal surface T, parallel to the fourth internal surface I₄ and opposite thereto. Moreover, the third portion 72 is traversed by a plurality of distal holes 74, of a through type.

The third and fourth internal surfaces I₃, I₄ delimit a recess 69, arranged inside which is the auxiliary device 70. In particular, the auxiliary device 70 is arranged substantially in the focus of the paraboloid of the third internal surface I₃, in contact with the fourth internal surface I₄, in such a way as to be spaced apart from the contact surface S by a distance greater than the distance between the contact surface S and the microphone 28. In this way, the auxiliary device 70 generates a noise signal of an electrical type substantially indicating the environmental noise, i.e., acoustic signals different from the acoustic signal coming from the area of the body in contact with the contact surface S. The processing unit 10 subtracts the noise signal from the auscultation signal so as to filter the environmental noise, thus improving the quality of the auscultation signal.

Finally, the shell 32 may have a shape different from what has been illustrated and described herein.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A mobile device comprising:
a first microphone configured to receive an acoustic signal from an area of a body during a detection period and to generate an electrical auscultation signal as a function of the acoustic signal;
a transmitter;
an accelerometer configured to generate an acceleration signal indicating acceleration of the mobile device; and
a processing unit coupled to the first microphone, the transmitter, and the accelerometer, the processing unit configured to receive the auscultation signal from the first microphone and transmit the auscultation signal to an external electronic device through the transmitter the processing unit being configured to receive the acceleration signal and generate, based on the acceleration signal, a position signal indicating a position of the mobile device during the detection period, and to transmit said position signal to the external electronic device through the transmitter.

2. The mobile device according to claim 1, wherein the first microphone is a MEMS microphone.

3. The mobile device according to claim 1, further comprising at least one from among: a temperature sensor, a pressure sensor, and a humidity sensor.

4. The mobile device according to claim 1, further comprising a gyroscope configured to generate an attitude signal indicating an attitude of the mobile device; and
wherein the processing unit is configured to transmit the attitude signal to the external electronic device through the transmitter.

5. The mobile device according claim 1, comprising a shell that delimits a first cavity and forms at least one through hole that places the first cavity in fluid communication with an environment outside of the mobile device, the first microphone being arranged within the first cavity.

6. The mobile device according to claim 5, wherein the shell forms an internal surface of the first cavity that delimits the first cavity and has at least one first focus, the first microphone being arranged substantially in the at least one first focus.

7. The mobile device according to claim 5, further comprising a second microphone, and wherein the shell forms a contact surface designed to contact said area of the body, the second microphone being arranged further away from the contact surface than the first microphone and being designed to generate a noise signal indicating environmental noise, the processing unit being configured to process the auscultation signal based on the noise signal.

8. The mobile device according to claim 7, wherein the shell forms an internal surface of the second cavity that delimits a second cavity and has at least one second focus, the second microphone being arranged substantially in the second focus and within the second cavity.

9. The mobile device according to claim 5, wherein the shell has a first recess and a second recess designed to house a first finger tip and a second finger tip, respectively, of a user.

10. An electronic stethoscope comprising:
a computing device configured to receive an auscultation signal; and
a mobile device configured to communicate with the computing device, the mobile device including:
a first microphone designed to receive an acoustic signal from an area of a body during a detection period and to generate an auscultation signal as a function of the acoustic signal;
a transmitter;
an accelerometer configured to generate an acceleration signal indicating acceleration of the mobile device; and
a processing unit coupled to the first microphone, the transmitter, and the accelerometer, the processing unit configured to receive the auscultation signal from the first microphone and transmit the auscultation signal to the computing device through the transmitter, the processing unit further configured to receive the acceleration signal and generate, based on the acceleration signal, a position signal indicating a position of the mobile device during the detection period, and to transmit said position signal to the computing device through the transmitter.

11. The electronic stethoscope according to claim 10, wherein the computing device is configured to display the position of the mobile device during the detection period based on the position signal.

12. The electronic stethoscope according to claim 10, wherein the computing device is a cellphone or a computer.

13. The electronic stethoscope according to claim 10 wherein the mobile device is configured to wirelessly communicate with the computing device.

14. The electronic stethoscope according to claim 10 wherein the mobile device is coupled to the computing device by a wire.

15. The electronic stethoscope according to claim 10 wherein the accelerometer is configured to generate a plurality of acceleration signals respectively indicating acceleration of the mobile device in different directions.

16. The electronic stethoscope according to claim 10 wherein the accelerometer is configured to generate a plurality of acceleration signals respectively indicating acceleration of the mobile device in different directions and provide the plurality of acceleration signals to the processing unit.

17. The electronic stethoscope according to claim 10 wherein the computing device is configured to store the auscultation signal.

18. The electronic stethoscope according to claim 10 wherein the computing device is configured to reproduce said acoustic signal based on the auscultation signal.
